(19) United States
(12) Patent Application Publication
(10) Pub. No.: US 2026/0141685 A1
(43) Pub. Date: May 21, 2026

(54) METHOD AND DEVICE FOR DOCUMENTING CARE MEASURES

(71) Applicant: FORBENCAP GmbH, Sonthofen (DE)

(72) Inventor: Robert Ludwig CONLE, Sonthofen (DE)

(21) Appl. No.: 19/390,985

(22) Filed: Nov. 17, 2025

(30) Foreign Application Priority Data
Nov. 21, 2024 (DE) 10 2024 134 273.0

(51) Int. Cl. G06V 10/764; G06V 10/774; G06V 10/776; G06V 10/82; G06V 20/52; G06V 40/20; G16H 15/00; G16H 40/20

(52) U.S. Cl. CPC G06V 10/764; G06V 10/774; G06V 10/776; G06V 20/52; G06V 40/20; G16H 15/00; G16H 40/20; G06V 10/82

(57) ABSTRACT

A method for the automated documentation of care measures for the care of a patient has the steps of: capturing image data of interactions between a caregiver and a patient using an image capture unit, processing the captured image data using an image processing algorithm for recognizing the interactions in the image data, and generating text and/or speech labels for the recognized interactions using a machine label generation learning model for the automated documentation of care measures using the generated text and/or speech labels.

### CROSS-REFERENCE TO RELATED APPLICATION

This application claims the benefit of Germany Application No. DE 10 2024 134 273.0, filed Nov. 21, 2024, which is incorporated herein by reference in its entirety.

### TECHNICAL FIELD

The invention relates to a method and a device for the automated documentation of nursing measures for the care of a patient.

### BACKGROUND

Patient care, especially in geriatric and nursing care, requires careful documentation of the care measures carried out. This documentation serves to ensure the quality and traceability of care and to provide legal protection for the nurse and the patient.

In practice, documentation is often done manually, which is time-consuming and takes up valuable nursing staff resources. In addition, digital approaches using tablets etc. are increasingly being used for care documentation, but these approaches still require significant human interaction.

The need for efficient and precise documentation is particularly important in view of the increasing demand for nursing staff and the existing staff shortage. Reducing the workload for nursing staff could help to focus more on the actual care.

### SUMMARY

It is a task of the invention to provide a method and/or a device for the automated documentation of care measures for the care of a patient.

The problem is solved by a method according to the features of patent claim 1. The problem is solved by a device according to the features of patent claim 10.

According to a preferred aspect, a method for the automated

documentation of care measures for the care of a patient is proposed, comprising the steps of: capturing image data of interactions between a caregiver and a patient using an image capture unit, processing the captured image data using an image processing algorithm for recognizing the interactions in the image data, and generating text and/or speech labels for the recognized interactions using a machine label generation learning model (also referred to herein as a machine learning model or model for short), in particular a transformer model.

The method involves capturing image data of interactions between a caregiver and a patient. This image data preferably documents typical care measures such as mobilization, personal hygiene, medication administration and/or wound care. The image recording unit can take the form of a portable camera, for example integrated into smart glasses, or as a stationary device arranged in the room. Alternatively, the image capture unit could also include depth cameras or multispectral cameras to capture additional details of the interactions.

Furthermore, the captured image data is processed, whereby an image processing algorithm is used to recognize the interactions contained in the image data. Such an algorithm can, for example, be based on methods such as segmentation, object recognition or pose estimation. Additionally or alternatively, approaches for motion analysis, activity recognition or anomaly detection could be integrated in order to identify complex care measures or detect critical situations.

In a further step of the process, text and/or voice labels are generated from the recognized interactions. This is done using a machine learning model, preferably a transformer model, which has previously been trained using annotated care protocols and image data. The labels preferably describe the care measures performed precisely and in a structured manner, particularly in chronological order, and can be output as text and/or in the form of audio. Alternatively or additionally, other learning models, such as recurrent neural networks (RNNs) or convolutional neural networks (CNNs), could also be used to handle specific tasks such as the analysis of temporal image sequences.

The proposed method offers numerous technical advantages. It reduces the manual effort required for nursing documentation, which gives nursing staff more time for direct patient care. The use of machine learning models ensures a high level of precision and consistency in the documentation, and the automatically generated data can preferably be stored in an audit-proof manner and checked as required. The flexibility of the process preferably enables adaptation to different care environments and/or scenarios, while the scalability supports implementation in facilities of various sizes, from nursing homes to hospitals. In addition, the ability to anonymize the data ensures that data protection requirements are met.

It is understood that the steps according to the invention as well as further optional steps do not necessarily have to be carried out in the order shown, but can also be carried out in a different order. Further intermediate steps may also be provided. The individual steps may also comprise one or more sub-steps without departing from the scope of the method according to the invention.

The machine learning model preferably generates labels automatically from image data by learning to link visual patterns, movements and contextual information with specific meanings during training. This process is preferably carried out in several interlinked steps. First, the captured image data could be pre-processed to make it consistent and easier for the model to process. This preferably includes adjusting the image size, normalizing colour spaces and removing image noise. For video data, sequences of frames could preferably be extracted in order to analyze temporal sequences. The machine learning model preferably has a language model, in particular a large language model.

The machine learning model may have a GPT (Generative Pre-trained Transformer), which is based on the Transformer architecture and is particularly well suited for processing text and generating natural-sounding speech. The machine learning model may also have a BERT (Bidirectional Encoder Representations from Transformers), which is also based on the Transformer architecture and is characterized by its ability to bidirectionally analyze contextual relationships in texts. Alternatively, the machine learning model can have a T5 (Text-to-Text Transfer Transformer), which also uses a transformer architecture and specializes in formulating each text processing task as a text-to-text problem.

Furthermore, the machine learning model can have an XLNet, which is based on a transformer architecture with autoregressive and autoencoder-like mechanisms and can better model contextual dependencies. Another possible model could be RoBERTa (Robustly Optimized BERT Approach), an optimized version of BERT that achieves higher performance through more extensive training on larger data sets. The model could also be an ALBERT (A Lite BERT), a lightweight and optimized version of BERT that requires less memory and is faster to train.

In addition, the machine learning model can feature an OpenAI Codex, a Transformer-based model specifically trained to process and generate code. For multimodal applications, a CLIP (Contrastive Language-Image Pre-training) model can be used, which is based on a Transformer architecture and combines text and image information to relate visual and linguistic input. Finally, a Transformer-XL could be used, an extended Transformer architecture that can model longer contextual dependencies.

The model preferably extracts visual features from the data. In the first layers of a neural network, for example in Convolutional Neural Networks (CNNs) or Transformer models, basic patterns such as edges, colors or textures can be recognized. Advanced layers preferably abstract these features further and identify more complex structures such as objects or specific actions, such as handing a care utensil or washing a patient.

Object recognition and scene analysis algorithms such as YOLO or Mask R-CNN could preferably be used to recognize and segment individual objects in the images or videos. This analysis preferably makes it possible to recognize specific nursing actions such as preparing an injection or repositioning a patient in their visual context. For dynamic scenes in which movements are crucial, the model could analyze movements across several frames. Preferably, optical flow algorithms or 3D CNNs could be used to identify activities such as raising a patient or applying a dressing.

In order to link the information in a meaningful way, the model preferably interprets the recognized patterns and movements in the context of the care action. Transformer models or recurrent neural networks (RNNs) could preferably be used, as they take into account temporal and spatial relationships. For example, the model could recognize whether the nurse is handing a patient a glass of water, taking a blood pressure, changing a dressing, etc., or whether they are performing another action. This contextual interpretation is preferably crucial for generating precise labels.

In the final step, the results of object recognition, movement analysis and context interpretation could preferably be compared with previously learned care protocols. Based on this analysis, the model could create labels that describe the recognized action or observation, such as “patient washed”, “medication administered” or “patient mobilized”.

The model can preferably generate these labels automatically because it has been trained on annotated datasets containing images and videos with precise descriptions. During the training process, it could learn how specific visual patterns, such as the movement of a hand or the position of a care utensil, correlate with specific care actions. In addition, Transformer models could preferably combine information from multiple sources, such as image data, movement patterns and environmental information, to provide a contextual interpretation of the scene.

A practical example of automatic label generation could preferably be the preparation of a syringe. A camera could record how a nurse prepares a syringe. The model could recognize the nurse, the syringe and the sequence of movements and preferably generate the label “medication syringe prepared”. In another scenario, the model could analyse the movements of the caregiver and the patient during mobilization and preferably generate the label “Patient lifted out of bed”. When checking a patient's vital signs, the model could also automatically recognize the care utensil, the position of the caregiver and the interaction to preferably generate the label “Blood pressure measured”.

In a further preferred aspect, an apparatus for automated documentation of nursing actions for caring for a patient is proposed, the apparatus comprising an evaluation and computing device adapted to perform the following steps: capturing image data of interactions between a caregiver and a patient using an image capture unit; processing the captured image data using an image processing algorithm to recognize the interactions in the image data; and generating text and/or speech labels for the recognized interactions using a machine learning model, in particular a transformer model.

The statements made for the method apply accordingly to the device. It is understood that linguistic modifications of features formulated in terms of the method can be reformulated for the device in accordance with standard linguistic practice, without such formulations having to be explicitly listed here.

In a further aspect, it is proposed that the method comprises an image capturing unit comprising a camera and/or smart glasses and/or other wearable device for capturing images.

The image recording unit preferably enables the recording of visual data of interactions between the caregiver and patient. The camera can be stationary or mobile, while wearable devices such as smart glasses increase the flexibility of care staff. Alternatively or additionally, other wearable devices such as body cameras or wearables with an integrated camera could also be used. These features preferably facilitate seamless integration into the caregiver's workflow without interfering with the care act. The use of wearable imaging units allows image data to be captured in close proximity to the care act, which increases the precision of the documentation. Smart glasses can also provide contextual information and take the caregiver's line of vision into account. The flexibility in the choice of image acquisition unit contributes to the adaptability of the method to different care environments. Portable devices minimize space requirements and maximize mobility.

In a further aspect, it is proposed that the method comprises an image capturing unit which is arranged, in particular, stationary or movable, in a room in which the patient is being cared for and/or on a body of the caregiver and/or on a body of the patient.

The arrangement of the image acquisition unit preferably determines the perspective and range of the recorded data. Stationary units are fixed in rooms such as patient rooms or care areas and enable continuous monitoring. Movable image recording units such as portable cameras can preferably be positioned flexibly. Alternatively or additionally, sensors could be attached directly to the patient's or carer's body to ensure personalized recording. Stationary units provide permanent, room-wide coverage, while wearable devices document specific details up close. Body-mounted cameras provide individualized perspectives and reduce the impact of environmental elements. The versatility of placement options preferably increases functionality in different care environments. Movable image capture units improve usability in changing scenarios.

In a further aspect, it is proposed that the image processing algorithm comprises image data segmentation and/or object detection and/or motion analysis and/or activity detection and/or face detection and/or anomaly detection and/or object tracking and/or action classification and/or multimodal image processing and/or feature extraction.

The image processing algorithm preferably analyzes the captured image data and extracts specific information. Segmentation preferably divides the image into relevant areas, while object recognition identifies specific care utensils or body parts. Motion and activity recognition are preferably used to analyze dynamic scenes, while facial recognition can identify the patient or detect emotions. Multimodal image processing preferably combines different types of data, such as RGB and depth data, for a more comprehensive analysis. These algorithms preferably enable precise recognition of care actions and improve the reliability of data analysis. The integration of multiple algorithms preferably offers high flexibility and adaptability to different scenarios.

In a further aspect, it is proposed that the image processing algorithm comprises a machine learning model for image processing, in particular a convolutional neural network.

Convolutional neural networks (CNNs) are particularly effective for image processing and can recognize patterns and objects in the image data. Alternatively, transformer models and/or hybrid approaches could also be used. The machine learning model preferably increases the accuracy and speed of image processing. The use of CNNs preferably enables efficient processing of large amounts of data and promotes automation.

In general, the image processing algorithm may comprise a machine learning model and/or a statistical model and/or an analytical model, in particular a hybrid model comprising several of the aforementioned models in combination.

In a further aspect, it is proposed that the machine label generation learning model is trained or at least fine-tuned using care protocols, a care history, the patient's care and associated image data.

Training the model on specific and/or predetermined maintenance protocols preferably enables application-specific customization. Alternatively, publicly available or synthetic data could be used to initialize the model. Training on specific data preferably increases accuracy and context sensitivity. Fine-tuning allows for personalized and context-dependent documentation.

In a further aspect, it is proposed that the generated text and/or speech labels are output as an audio and/or text file, in particular an editable audio and/or text file.

The output in various formats enables simple integration into existing systems. Alternatively, labels can be displayed visually in dashboards. Such flexibility in the output preferably promotes usability and adaptability. The editable output preferably facilitates corrections and/or adjustments.

In a further aspect, it is proposed that erroneous labels are identified in a revision of the care protocols and subsequently used to retrain the machine label generation learning model and/or the image processing algorithm.

The continuous improvement of the model through faulty labels preferably promotes the ability to learn. Alternatively, external sources of feedback could be included. Post-training increases long-term accuracy and robustness. The system remains adaptive and adapts to changing conditions.

In a further aspect, it is proposed that a computer program product comprises instructions which, when the program is executed by a computer, cause the computer to perform the steps of the present method according to any embodiment.

The computer program product forms the basis for implementing the methods. Availability as a software product facilitates distribution and integration. Simple use on existing hardware is made possible.

A computer program that implements the steps of a method for the automated documentation of care measures can preferably have a modular structure and consist of several components that preferably perform specific tasks. For example, it could consist of the following modules:

An input module is preferably used to integrate the image acquisition unit, which is either portable, stationary or mobile. This input module preferably controls the acquisition of the image data, synchronizes the images with other data sets (e.g. time stamps or patient information) if required and/or prepares the image data for processing. In addition, this input module can preferably apply filters to optimize image quality and integrate privacy-friendly techniques such as anonymization or blurring of facial data.

An image processing module preferably analyzes the captured image data using one or more image processing algorithms. Neural networks such as convolutional neural networks (CNNs) or vision transformers (ViTs) could be used for object recognition, segmentation, motion analysis or activity recognition. This image processing module is preferably designed to extract relevant information such as the caregiver's actions, the patient's condition or the use of care utensils.

A label generation module preferably processes the results of the image processing module and creates text and/or speech labels. This label generation module preferably uses a machine learning model, such as a transformer model, which has been trained on annotated care protocols and image data. The labels are preferably generated in a structured form and can be formatted as text or audio output as required. Furthermore, the label generation module may preferably include a feedback component that identifies erroneous labels and uses them to optimize the machine learning model.

An output module preferably takes care of the storage and output of the generated labels. It could provide the labels in various formats, such as editable text files, audio formats or in the form of a visual user interface, such as a dashboard that displays real-time updates. This output module can also provide an interface for integration with external systems, such as electronic health records (EHR).

A training and adaptation module preferably enables the further development and fine-tuning of the models used. It could carry out retraining on the basis of new data or incorrect labels and thus continuously improve the precision and robustness of the system. It could work both online (during use) and offline (on prepared data sets).

A security module preferably ensures that data protection requirements are met. It could use encryption techniques to secure the stored data and restrict access to the data to authorized users. This module can also integrate mechanisms to anonymize sensitive information such as faces or personal details of the patient.

In a further aspect, it is proposed that a computer-readable data carrier stores the present computer program product.

The data carrier is preferably used for long-term storage and distribution of the program. Storage on data carriers preferably ensures portability. Flexible distribution and backup are made possible.

A computer-readable data carrier on which such a computer program is stored can be designed in a variety of ways. A typical data carrier could be a physical medium such as a CD, DVD or Blu-ray disc on which the program is permanently stored. Alternatively, a flash-based storage medium such as a USB stick or SSD could be used, which offers greater storage capacity and easier handling.

Another approach would be a cloud-based data carrier, where the program data is stored on a remote server and can be accessed via the internet. Such solutions are particularly suitable for scenarios in which updates and shared access to the program are required. In all cases, the data carrier could provide additional security mechanisms such as password protection or encryption to prevent unauthorized access.

The data carrier can also be designed to be directly compatible with existing systems, such as hospital information systems, and enable seamless integration. This promotes easy distribution and scalability of the program in different care environments.

The training of the machine learning model for the present method can preferably take place in several phases in order to flexibly meet the requirements of automated care documentation. First, a basic model of the machine learning model is preferably provided, which is trained on general data before being fine-tuned using specific care data. This process preferably includes the phases of data collection, data preparation, model training, validation and optimization to ensure high accuracy and robustness.

The training data for the machine learning model can preferably come from a variety of sources, such as video and image recordings of real care interactions, annotated care protocols or synthetically generated data. This data could include different care actions, such as personal care, mobilization, medication administration or wound care, and could be supplemented by metadata such as timestamps, care environment features or sensor information (e.g. depth data). The training data can preferably be in formats such as MP4 or AVI for video data, JPEG or PNG for images and CSV or JSON for associated annotations. Annotations could contain labels that assign semantic meanings to the data, such as “mobilization of the patient” or “administration of medication”.

The training data is preferably processed by the machine learning model by means of pre-processing, which includes normalization of the image data (e.g. adjustment of resolution and colour values), extraction of relevant features (e.g. movement patterns or object contours) and segmentation of the scenes. The annotated labels are preferably used as target values for training the model. During the training process, the model gradually abstracts the features in order to learn the relationships between the inputs (image data) and the outputs (labels).

A foundation model approach could preferably be used to increase efficiency. A large pre-trained model is used, preferably trained on extensive, general data sets such as large video or image databases. This foundation model preferably has a broad understanding of general visual and semantic patterns and reduces the need for extensive maintenance-specific data. Subsequently, the foundation model is preferably fine-tuned with domain-specific care data, such as annotated videos from nursing homes or hospitals, to cover the specific requirements of care documentation.

An application example of the present method could preferably be implemented in a hospital for the documentation of mobilization measures for bedridden patients. In such a scenario, a stationary camera installed in the patient's room could preferably record the interactions between the caregiver and the patient. The system preferably records scenes such as moving the patient in bed, sitting them up or transferring them to a wheelchair. The camera could preferably transmit the data to the image processing module, which analyzes the scenes in real time and recognizes relevant movement patterns. A machine learning model, which has preferably been specially trained on mobilization data, could then generate text and/or voice labels such as “Patient repositioning completed” or “Patient transferred to wheelchair”.

The generated labels could preferably be automatically integrated into the hospital's electronic patient record system. At the same time, a dashboard interface could enable the nursing staff to check the documentation in real time and edit it if necessary. Incorrect labels could preferably be corrected manually, with these corrections preferably feeding into the feedback loop of the machine learning model to continuously optimize the machine learning model. This example shows how the process can preferably be used efficiently in order to reduce the documentation effort, increase accuracy and reduce the workload of nursing staff.

The machine learning model can be further enhanced by the use of additional audio data, preferably captured by a microphone, as this provides an additional dimension of contextual information. Audio data could preferably include speech, environmental sounds and/or specific acoustic events such as the rustling of clothing, the opening of packages and/or the sound of a wheelchair. This information can preferably be captured by a microphone that is either integrated into the image capture unit, such as smart glasses or a stationary camera, or used as a separate device. The synchronization of audio and image data could preferably establish temporal correspondences between visual and acoustic signals. For example, the machine learning model could preferably recognize that an item of clothing is being put on by hearing the sound of a zipper in combination with a visual action.

Speech recognition and/or analysis could preferably be integrated into the system to transcribe spoken words or phrases during care. This can preferably provide cues to the action being performed, such as when the caregiver says, “I'm going to take your blood pressure now.” Such linguistic cues could preferably complement the visual analysis and support the model in correctly interpreting the action. In addition, ambient sounds such as the clicking of a blood pressure monitor, the hissing of an oxygen mask or the rushing of water could be analyzed. These sounds could preferably serve as indicators for certain care actions, such as washing hands or putting on equipment.

The combination of visual and audio data through multimodal data fusion can preferably enable a holistic analysis of care actions. Transformer models or other specialized architectures for multimodal data could preferably be used to merge visual and audio information. This fusion preferably allows the model to supplement unclear information from one data stream with the other. Training data could preferably be augmented with annotated audio clips containing, for example, typical sounds and speech patterns during care. This preferably sensitizes the model to acoustic variations such as different pitches and/or dialects.

A practical example shows the advantages of this integration. Suppose a nurse measures a patient's blood pressure. The visual model could preferably recognize the caregiver and the blood pressure monitor, but might not be able to clearly classify the specific action. By integrating audio data, the model could preferably hear the hissing of air as the device is inflated and the nurse saying “I'm checking your blood pressure now”. This information preferably enables the model to generate the label “Blood pressure measurement performed” with high precision.

This integration of audio data could preferably make the machine learning model more accurate and flexible, especially in situations where the view of the action is limited and/or visual data alone is not sufficient. The combination of visual and acoustic signals preferably improves contextualization and enables faster, more reliable and more comprehensive automatic documentation of care actions.

In a further aspect, data security and anonymity of the patient can preferably be ensured by a number of technical measures. The captured image data of interactions between a caregiver and a patient can preferably be anonymized before processing by the image processing algorithm. Preferably, methods such as facial recognition and masking could be used to automatically recognize faces and/or other identifiable features and make them unrecognizable by blurring, pixelation or complete coverage.

To prevent unauthorized access, the image data could preferably be secured by end-to-end encryption during transmission from the image acquisition unit to the evaluation and computing unit. An encryption method such as AES-256 could preferably be used to ensure that only authorized systems can decrypt and further process the data. In addition, pseudonymized data structures could be used in which personal identifiers, such as the patient's name, are replaced by unique, untraceable codes.

The data could preferably be stored on local, protected servers that are physically and digitally secured against attacks. Alternatively, edge computing solutions could preferably be used in which the processing and anonymization of the data already takes place on the image acquisition unit or in a local unit so that no sensitive data has to be transferred to external networks. Alternatively, the captured data can also be deleted after the report has been visualized.

To ensure data security, access control systems could preferably be implemented that only allow authorized persons access to certain data areas. These systems could preferably be based on two-factor authentication (2FA) and/or biometric methods. In addition, all data operations could preferably be logged by an audit logging system in order to identify and, if necessary, block suspicious access attempts.

Finally, a privacy-centric approach could be complemented by the use of differential privacy, where noise signals are added to the data to prevent inferences about individual patients while preserving the useful information for processing. These technical measures could preferably ensure secure and anonymized processing of the data in the context of the claimed method.

The embodiments and further developments described can be combined with each other as desired.

Other possible embodiments, further embodiments and implementations of the invention also include combinations of features of the invention described above or below with respect to the embodiment examples that are not explicitly mentioned.

### BRIEF DESCRIPTION OF DRAWINGS

The accompanying drawings are intended to provide a further understanding of embodiments of the invention. They illustrate embodiments and, in connection with the description, serve to explain principles and concepts of the invention.

Other embodiments and many of the advantages mentioned are shown in the drawings. The elements shown in the drawings are not necessarily shown to scale in relation to each other.

FIG. 1 shows a schematic flow diagram of an embodiment of the process.

FIG. 2 shows a schematic view of a block diagram of the present device.

FIG. 3 shows a schematic view of a block diagram of the present device.

### DETAILED DESCRIPTION

In the figures in the drawings, identical reference symbols denote identical or functionally identical elements, parts or components, unless otherwise indicated.

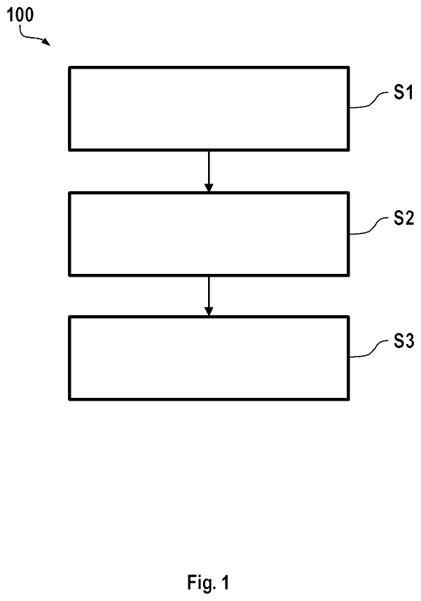

FIG. 1 shows a schematic flow diagram of an embodiment of a present method for the automated documentation of nursing measures for the care of a patient.

In any embodiment, the method can be carried out at least in part by a device 100, which for this purpose can comprise several components not shown in more detail, for example one or more provision devices and/or at least one evaluation and computing device. It is understood that the provision device may be formed together with the evaluation and computing device, or may be different from the latter. Furthermore, the device 100, which may be part of a system, may comprise a storage device and/or an output device and/or a display device and/or an input device.

The computer-implemented method comprises at least the following steps:
In a step S1, image data of interactions between a caregiver and a patient is captured using an image capture unit.
In a step S2, the captured image data is processed using an image processing algorithm to recognize the interactions in the image data.
In a step S3, text and/or speech labels are generated for the recognized interactions using a machine learning label generation model for the automated documentation of care measures using the generated text and/or speech labels.

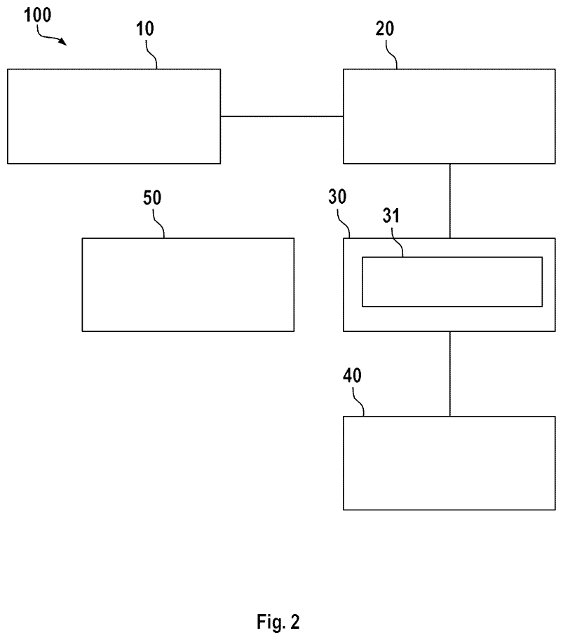

FIG. 2 shows a schematic representation of the architecture of the device 100 for the automated documentation of care measures. The device 100 includes an image capture unit 10 that captures visual data of interactions 130 between a caregiver 110 and a patient 120. The image capture unit 10 may include various embodiments, such as a stationary installed camera, a wearable camera or a camera integrated into (smart) glasses. The image capturing unit 10 may also be arranged on a head of the caregiver 110. The captured data is preferably forwarded to an evaluation and computing unit 20 via an interface.

The evaluation and computing unit 20 analyzes the image data and preferably comprises various modules. First, the data is processed by an image processing module 25, which extracts visual features, such as the position of the caregiver 110, the patient 120 and any care utensils. The processed image data is then analyzed in the label generation module 30, which automatically generates text and/or speech labels using a machine learning model 31. These labels describe the identified care actions, such as “Patient mobilized” or “Blood pressure measurement performed”.

The label generation module 30 preferably forwards the generated labels to an output module 40, which preferably provides the output in different formats. The output module 40 preferably enables the generation of text documents that can be integrated directly into electronic patient records and/or the output of voice information to support the caregiver. A processing unit 50, in particular a central processing unit, preferably controls the entire data flow between the individual modules and preferably ensures that the data is processed and forwarded consistently. The architecture enables automated, precise and efficient documentation of care actions.

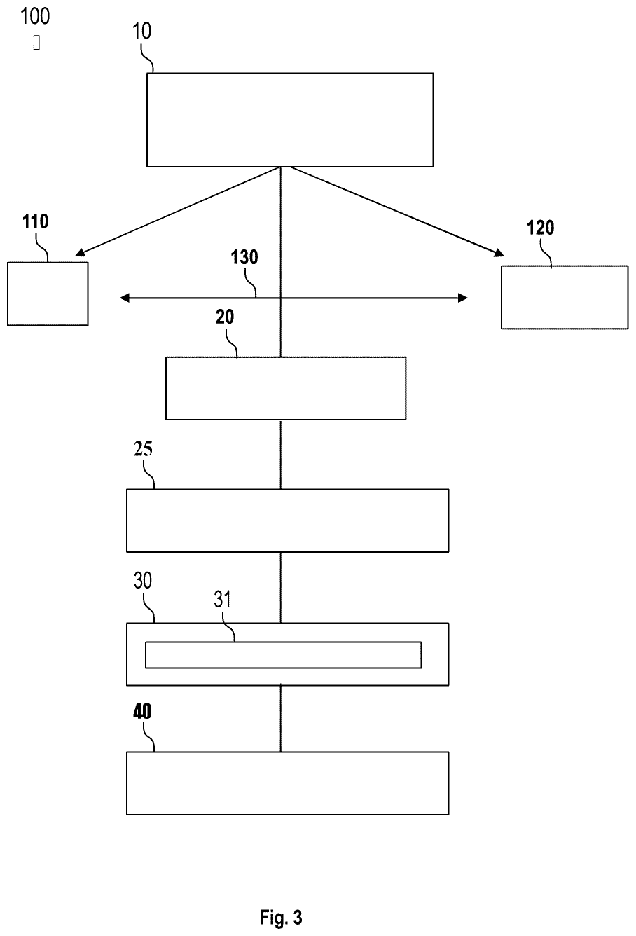

FIG. 3 shows the spatial arrangement of the individual system components in a typical care environment. In the center of the illustration, a patient 120 is schematically shown being cared for by a caregiver 110. The interactions 130 between the caregiver 110 and the patient 120 are captured by an image capture unit 10. This image recording unit 10 can be designed as a portable component, for example in the form of smart glasses, or as a stationary camera installed in a patient room.

The image recording unit 10 records the visual data of the interactions 130 and preferably synchronizes it with additional information, such as movement or environmental data. The recorded data is transmitted to the evaluation and computing unit 20, which analyzes, for example, movements, objects and/or actions. For example, it can be recognized whether the caregiver 110 is repositioning the patient 120, administering a medication and/or measuring vital parameters.

After analyzing the interactions 130 in the evaluation and computing unit 20, the results are preferably forwarded to the label generation module 30. The label generation module 30 uses the at least one machine learning model or a model composition of multiple machine and/or statistical and/or analytical models to translate the data into, in particular, meaningful labels for documenting the care of the patient 120. These labels could be, for example, “patient washed”, “dressing changed” or “medication given”. The labels are preferably created automatically in the context of the care action and transmitted to the output module 40. The output module 40 then preferably outputs the care documentation in a structured form using the generated labels. Preferably, general context information and/or template information on the structure and/or type of documentation can also be generated on the basis of language processing and/or using a language model. This output is either in the form of a text document that is stored in a digital patient file, for example, and/or as a voice file that provides auditory support for nursing staff.

### LIST OF REFERENCE SYMBOLS

10 Image acquisition unit
20 Evaluation and calculation unit
25 Image processing module
30 Label generation module
31 Machine learning model
40 Output module
50 Process unit
100 Device
110 Caregiver
120 Patient
130 interactions
S1 Step 1 (capturing image data)
S2 Step 2 (Processing image data)
S3 Step 3 (Generating labels)

1. A method for automated documentation of nursing methods for the care of a patient, comprising:
capturing image data of interactions between a caregiver and a patient using an image capture unit;
processing the captured image data using an image processing algorithm configured to recognize the interactions in the image data; and
generating text and/or speech labels for the recognized interactions using a machine learning label generation model, wherein the text and/or speech labels document care actions.

2. The method of claim 1, wherein the image capture unit comprises a camera, smart glasses, or another portable device for capturing images.

3. The method of claim 1, wherein the image capture unit is arranged in a room in which the patient is being cared for, on the body of the caregiver, or on the body of the patient.

4. The method of claim 1, wherein the image processing algorithm comprises one or more of the following: image data segmentation, object detection, motion analysis, activity detection, face detection, anomaly detection, object tracking, action classification, multimodal image processing, or feature extraction.

5. The method of claim 1, wherein the image processing algorithm comprises a machine learning model for image processing.

6. The method of claim 1, wherein the machine learning label generation model is trained or fine-tuned using care protocols, care history data, and image data associated with the patient.

7. The method of claim 1, wherein the generated text and/or speech labels are output as an audio file, a text file, or both.

8. The method of claim 1, further comprising identifying erroneous labels in a revision of care protocols and using the identified erroneous labels to retrain the machine learning label generation model or the image processing algorithm.

9. A computer program product comprising instructions which, when executed by a computer, cause the computer to perform the method of claim 1.

10. A device for automated documentation of care measures for the care of a patient, comprising:
an image capture unit configured to capture image data of interactions between a caregiver and a patient;
an evaluation and computing device configured to process the captured image data using an image processing algorithm to recognize the interactions in the image data; and
a machine learning model configured to generate text and/or speech labels for the recognized interactions, wherein the labels document the care actions.
